(19) United States
(12) Patent Application Publication
(10) Pub. No.: US 2023/0082422 A1
(43) Pub. Date: Mar. 16, 2023

(54) PHYSIOLOGICAL INFORMATION ACQUISITION APPARATUS, PROCESSING DEVICE, AND NON-TRANSITORY COMPUTER READABLE STORAGE MEDIUM

(71) Applicant: NIHON KOHDEN CORPORATION, Tokyo (JP)

(72) Inventors: Takuya UMEMOTO, Tokorozawa-shi (JP); Jun Motogi, Tokorozawa-shi (JP); Keiichi Sato, Tokorozawa-shi (JP); Wataru Matsuzawa, Tokorozawa-shi (JP)

(73) Assignee: NIHON KOHDEN CORPORATION, Tokyo (JP)

(21) Appl. No.: 17/901,032

(22) Filed: Sep. 1, 2022

(30) Foreign Application Priority Data
Sep. 10, 2021 (JP) 2021-147667

(51) Int. Cl. A61B 5/00

(52) U.S. Cl. CPC A61B 5/7275; A61B 5/7264; A61B 5/7246

(57) ABSTRACT

A physiological information acquisition apparatus that acquires physiological information of a subject includes a reception device configured to receive waveform data corresponding to a measurement waveform of the physiological information from a sensor, and to acquire values of a plurality of characteristic parameters associated with the measurement waveform based on the waveform data, a processing device configured to input the values of the plurality of characteristic parameters to a machine-learned model to acquire a prediction result for at least one of a plurality of classes into which the waveform data is classified, and to specify a level of importance of each of the plurality of characteristic parameters for the prediction result, and an output device configured to output an index indicating a name of at least one of the plurality of characteristic parameters and the level of importance specified for the at least one characteristic parameter.

### CROSS-REFERENCE TO RELATED APPLICATIONS

The present application claims priority from Japanese Patent Application No. 2021-147667, filed on Sep. 10, 2021, the entire content of which is incorporated herein by reference.

### TECHNICAL FIELD

The presently disclosed subject matter relates to a physiological information acquisition apparatus that acquires physiological information of a subject. The presently disclosed subject matter also relates to a processing device that processes physiological information of a subject, and a non-transitory computer readable storage medium that stores a computer program executable by a processing unit mounted on the processing device.

### BACKGROUND

JP-A-2009-100934 discloses an apparatus that acquires a pulse wave, which is an example of physiological information of a subject. When determining that noise of a predetermined level or more is mixed in a measurement waveform of the pulse wave, the apparatus notifies a user of this.

### SUMMARY

An object of the presently disclosed subject matter is to improve interpretability of a processing result of physiological information acquired from a subject.

According to a first aspect of the presently disclosed subject matter, there is provided a physiological information acquisition apparatus that acquires physiological information of a subject, the apparatus including:
a reception device configured to receive waveform data corresponding to a measurement waveform of the physiological information from a sensor, and to acquire values of a plurality of characteristic parameters associated with the measurement waveform based on the waveform data;
a processing device configured to input the values of the plurality of characteristic parameters to a machine-learned model to acquire a prediction result for at least one of a plurality of classes into which the waveform data is classified, and to specify a level of importance of each of the plurality of characteristic parameters for the prediction result; and
an output device configured to output an index indicating a name of at least one of the plurality of characteristic parameters and the level of importance specified for the at least one characteristic parameter.

According to a second aspect of the presently disclosed subject matter, there is provided a processing device that processes physiological information of a subject, the device including:
an interface configured to receive values of a plurality of characteristic parameters acquired based on waveform data corresponding to a measurement waveform of the physiological information, the plurality of characteristic parameters being associated with the measurement waveform; and
one or more processors configured to input the values of the plurality of characteristic parameters to a machine-learned model to acquire a prediction result of at least one of a plurality of classes into which the waveform data is classified, specify a level of importance of each of the plurality of characteristic parameters for the prediction result, and output, to an output device, an index indicating a name of at least one of the plurality of characteristic parameters and the level of importance specified for the at least one characteristic parameter.

According to a third aspect of the presently disclosed subject matter, there is provided a non-transitory computer readable storage medium that stores a computer program executable by one or more processors mounted on a processing device that processes physiological information of a subject, the computer program causing the processing device to execute processing of:
receiving values of a plurality of characteristic parameters acquired based on waveform data corresponding to a measurement waveform of the physiological information, the plurality of characteristic parameters being associated with the measurement waveform,
inputting the values of the plurality of characteristic parameters to a machine-learned model to acquire a prediction result for at least one of a plurality of classes into which the waveform data is classified,
specifying a level of importance of each of the plurality of characteristic parameters for the prediction result, and
outputting, to an output device, an index indicating a name of at least one of the plurality of characteristic parameters and the level of importance specified for the at least one characteristic parameter.

### BRIEF DESCRIPTION OF DRAWINGS

FIG. 1 illustrates a functional configuration of an electrocardiograph according to an embodiment;

FIG. 2 illustrates a process executed by a processing device in FIG. 1;

FIG. 3 illustrates an example of indices output by an output device in FIG. 1;

FIG. 4 illustrates another example of the indices output by the output device in FIG. 1;

FIG. 5 illustrates another example of the indices output by the output device in FIG. 1;

FIG. 6 illustrates another example of the indices output by the output device in FIG. 1;

FIG. 7 illustrates another example of the indices output by the output device in FIG. 1; and

FIG. 8 illustrates another example of the indices output by the output device in FIG. 1.

### DESCRIPTION OF EMBODIMENTS

An example of an embodiment will be described in detail below with reference to the accompanying drawings.

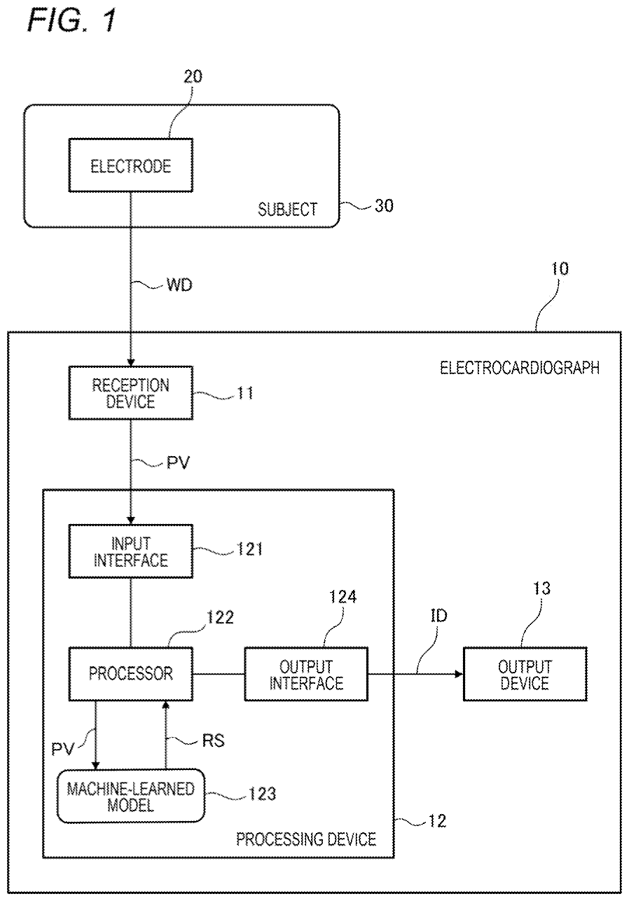

FIG. 1 illustrates a functional configuration of an electrocardiograph 10 according to the embodiment. The electrocardiograph 10 is an apparatus that acquires an electrocardiogram of a subject 30 through an electrode 20. The electrocardiogram is an example of physiological information. The electrocardiograph 10 is an example of a physiological information acquisition apparatus. The electrode 20 is an example of a sensor.

The electrocardiograph 10 includes a reception device 11. The reception device 11 receives waveform data WD corresponding to a measurement waveform of an electrocardiogram from the electrode 20. The waveform data WD may be in a form of analog data or in a form of digital data. The reception device 11 may include reception control circuitry that perform reception control. When the waveform data WD is in the form of analog data, the reception device 11 includes an appropriate conversion circuit including an A/D converter.

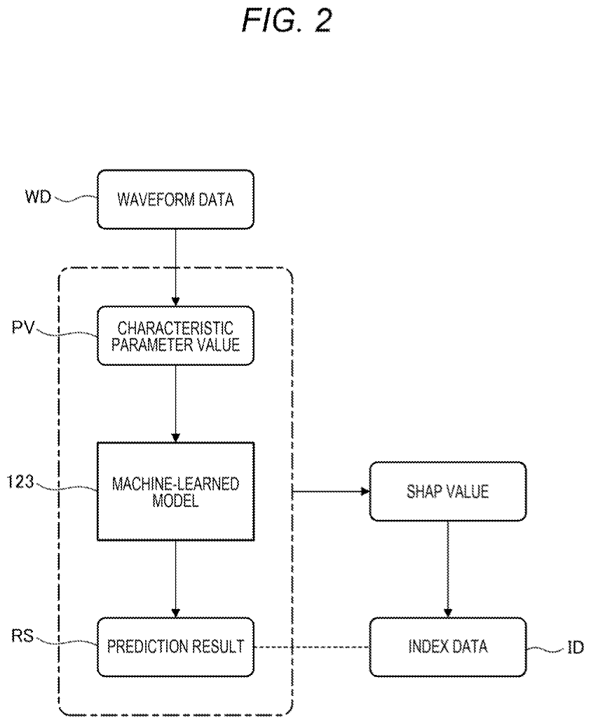

As illustrated in FIG. 2, the reception device 11 acquires values PV of a plurality of characteristic parameters associated with the measurement waveform of the electrocardiogram based on the waveform data WD. The characteristic parameters are data representing shape characteristics of the measurement waveform. Examples of the characteristic parameters of the measurement waveform of the electrocardiogram include the polarity and the amplitude of a P-wave, the width of a QRS-wave, the polarity and the amplitude of a T-wave, the polarity and the amplitude of a U wave, the width of the P-wave, the width of a Q-wave, the width of an R-wave, the width of an S-wave, an interval between the P-wave and the Q-wave, an interval between the Q-wave and the T-wave, an interval between the P-wave and the P-wave, and an interval between the R-wave and the R-wave.

Specifically, an average waveform is calculated based on a general electrocardiogram measurement algorithm for each lead based on the waveform data WD obtained within predetermined time, and the values of the characteristic parameters are acquired for the average waveform.

As illustrated in FIG. 1, the electrocardiograph 10 includes a processing device 12. The processing device 12 includes an input interface 121, a processor 122, a machine-learned model 123, and an output interface 124.

The input interface 121 receives the values PV of a plurality of characteristic parameters acquired by the reception device 11.

As illustrated in FIG. 2, the processor 122 inputs the values PV of a plurality of characteristic parameters received by the input interface 121 to the machine-learned model 123.

The machine-learned model 123 is an algorithm that predicts, as an output, the presence or absence of a risk of paroxysimal atrial fibrillation (pAF) for the subject 30 based on the input values PV of a plurality of parameters. A prediction result that “there is a risk of pAF” and a prediction result that “there is no risk of pAF” are examples of a plurality of classes into which the waveform data WD is classified.

A prediction result RS of the machine-learned model 123 may have various forms. Only a classification result related to a single class of “there is a risk of pAF” may be output, or the classification result related to the single class may be output together with a potential that “the risk of pAF is 84%”. Alternatively, a classification result related to a plurality of classes of “the potential of pAF is 84%” and “the potential of not pAF is 16%” may be output together with potentials.

The machine-learned model 123 is a prediction algorithm generated by machine learning. The machine-learned model 123 may be generated by machine learning using a neural network such as deep learning, or by another machine learning algorithm. Examples of the other machine learning algorithm include a decision tree, a random forest, and a support vector machine.

The processor 122 specifies the level of importance of each of the values PV of a plurality of characteristic parameters for the prediction result RS. In this example, the processor 122 calculates a SHapley Additive exPlanations (SHAP) value for each of the values PV of a plurality of characteristic parameters. That is, the SHAP value is an example of the level of importance.

The SHAP value is acquired by calculating how the prediction result changes due to the presence of certain characteristic amount (characteristic parameter) for all combinations of characteristic amounts that do not include the certain characteristic amount. When the prediction result greatly changes due to the presence of the certain characteristic amount, the SHAP value increases. In this case, the characteristic amount can be determined to greatly contribute to the prediction. That is, by calculating the SHAP value for each of a plurality of characteristic parameters, it is possible to specify the level of contribution (level of importance) of each characteristic parameter to the prediction result RS. A method itself for calculating the SHAP value is well known, and thus a detailed description thereof will be omitted.

The processor 122 generates index data ID for outputting an index indicating a name of at least one of a plurality of characteristic parameters and the SHAP value specified for the at least one characteristic parameter, and outputs the index data ID from the output interface 124. The index data ID may be in a form of analog data or in a form of digital data. When the index data ID is in the form of analog data, the output interface 124 includes an appropriate conversion circuit including a D/A converter.

As illustrated in FIG. 1, the electrocardiograph 10 includes an output device 13. The output device 13 may be implemented as a display that displays an index based on the index data ID output from the processing device 12. The display of the index is an example of the output of the index. The output mode of the index may be other visual presentation such as projection or printing of an image, and may also be auditory presentation in addition to or instead of visual presentation. The output device 13 may include output control circuitry that perform output control.

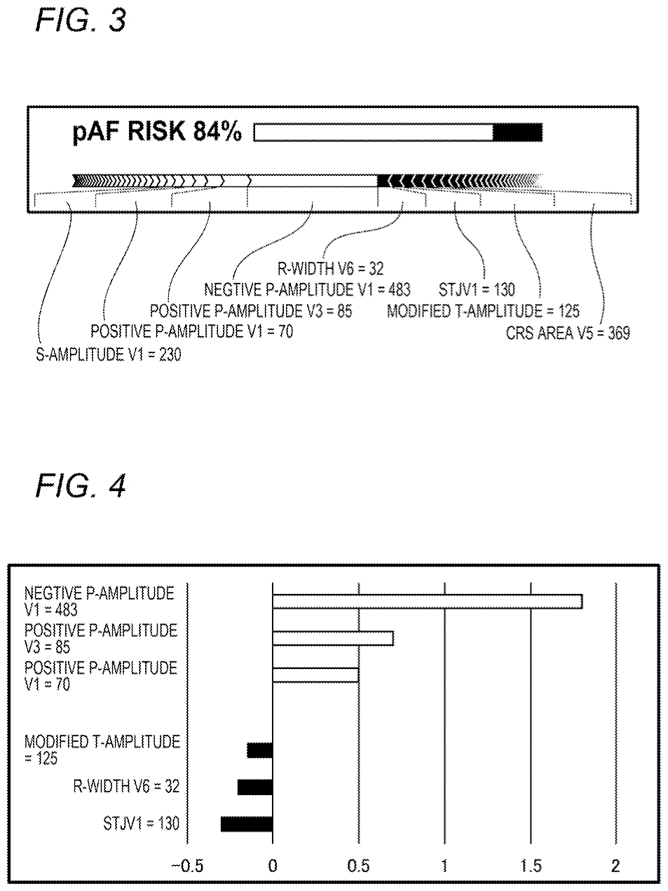

FIG. 3 illustrates an example of indices output by the output device 13. In the indices according to this example, the class of “there is a risk of pAF” included in the prediction result is indicated together with the potential. In addition, in the indices according to this example, names of a plurality of characteristic parameters of the measurement waveform of the electrocardiogram are indicated together with the SHAP value specified for each characteristic parameter and a measurement value of each characteristic parameter. In this example, the magnitude of the SHAP value corresponds to the length of a bar extending in a left-right direction.

In this example, it is illustrated that “area below negative P-waveform of lead V1” in the measurement waveform of the electrocardiogram contributed most greatly to the prediction result that “the potential of pAF is 84%”. On the other hand, it is illustrated that “R-wave width of lead V6” in the measurement waveform of the electrocardiogram contributed most greatly to the prediction result that “pAF may not occur”.

When non-structured data such as the waveform data WD corresponding to the measurement waveform of the electrocardiogram is a target of prediction processing, it is generally difficult to visualize the basis of prediction. However, according to the configuration as described above, by using a method of visualizing a characteristic parameter that can be handled as structured data, it is possible for a user to recognize by the index which characteristic parameter in the measurement waveform of the electrocardiogram contributes to the prediction. Therefore, it is possible to improve the interpretability of a processing result of the electrocardiogram.

In particular, when the machine-learned model 123 is generated by machine learning using a neural network, it is theoretically difficult to obtain a clear basis of a prediction result. In the medical field, an ambiguous basis of determination tends to be avoided, and therefore, in such a case, the effect of improving the interpretability of the processing result of the electrocardiogram is more remarkable.

In the indices according to this example, a plurality of SHAP values specified for a plurality of characteristic parameters are illustrated in a state in which relative levels of importance can be compared. For example, regarding the prediction result that “the potential of pAF is 84%”, it is illustrated that “area above negative P-waveform of lead V1” in the measurement waveform of the electrocardiogram has a higher level of importance than “positive P-wave amplitude of lead V3”.

According to such a configuration, it is possible for the user to recognize by the index which characteristic parameter of the measurement waveform of the electrocardiogram contributes more greatly to the prediction than which characteristic parameter. Therefore, it is possible to further improve the interpretability of the processing result of the electrocardiogram.

As long as the plurality of SHAP values specified for a plurality of characteristic parameters can be indicated in a state in which the relative levels of importance can be compared, the indices output by the output device 13 may also take a mode as illustrated in FIG. 4. In this example, names of the plurality of characteristic parameters of which levels of importance are specified are arranged in an upper-lower direction. The magnitude of the SHAP value specified for each characteristic parameter corresponds to the length of a bar arranged on the side of the name.

On the other hand, the index output by the output device 13 may indicate the level of importance of a single characteristic parameter. For example, the name of the characteristic parameter that contributes most greatly to the prediction result may be indicated.

In this case, the visibility of the index can be improved, and it is possible for the user to recognize information having a relatively high level of importance.

For example, in the indices illustrated in FIG. 3, the SHAP value specified for each characteristic parameter is illustrated together with the value of the characteristic parameter. For example, it is indicated that “positive P-wave amplitude of lead V3” is 85 mV and “positive P-wave amplitude of lead V1” is 70 mV.

In general, a medical worker recognizes a value that may be taken by a characteristic parameter. Therefore, according to the configuration as described above, it is possible to consider the validity of the level of importance of a specific characteristic parameter for the prediction result indicated by the index while considering the value of the characteristic parameter.

However, the display of the value of at least one of a plurality of characteristic parameters may be omitted appropriately in view of the visibility of the index.

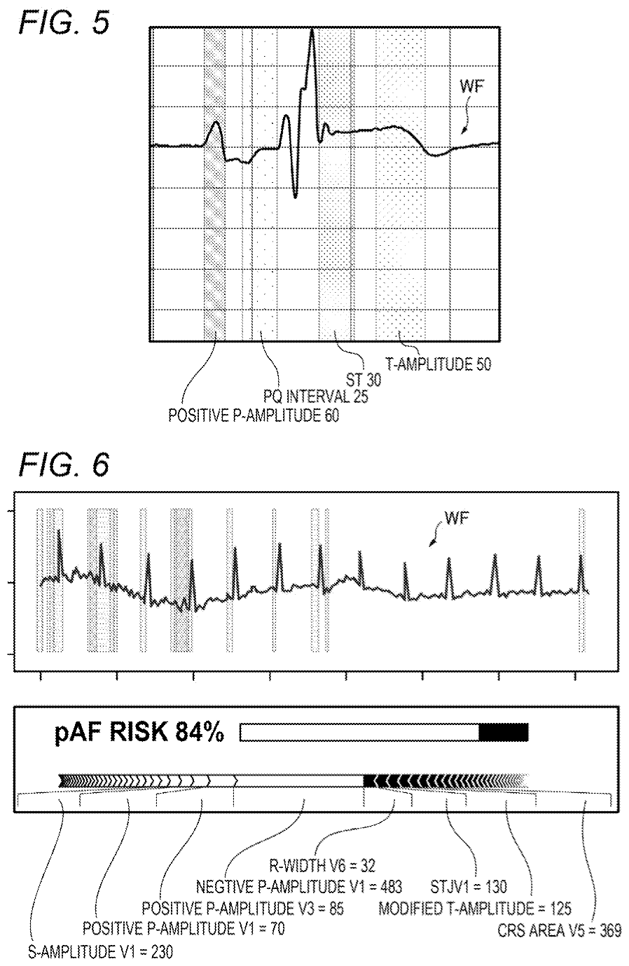

As illustrated in FIG. 5, the output device 13 may display an index indicating the name of at least one of a plurality of characteristic parameters and the SHAP value specified for the at least one characteristic parameter in a manner of overlapping the measurement waveform WF of the electrocardiogram.

In this example, the average waveform of the electrocardiogram acquired by the reception device 11 is displayed as the measurement waveform WF. In addition, indices including a plurality of characteristic parameter names including “positive P-wave amplitude”, “interval between P-wave and Q-wave”, “ST increase”, and “T-wave amplitude” for the prediction result that “pAF may occur” and the level of importance specified for each characteristic parameter are displayed in a manner of overlapping the measurement waveform WF.

More specifically, an index indicating the level of importance specified for a certain characteristic parameter is displayed as a band overlapping a portion of the measurement waveform WF with which the characteristic parameter is associated. A numerical value displayed in a manner of overlapping the band represents the value of the characteristic parameter. For example, the index related to “positive P-wave amplitude” is displayed together with “60 (mV)”, which is the value of “positive P-wave amplitude”, as a band overlapping the position of the P-wave in the measurement waveform WF. In this example, the magnitude of the SHAP value corresponds to the depth of the color of the band.

According to such a configuration, it is possible to intuitively grasp which characteristic parameter related to a portion of the measurement waveform WF contributed to the prediction result of the electrocardiogram.

When a plurality of characteristic parameters are associated with a specific portion of the measurement waveform WF and a plurality of levels of importance are specified for the plurality of characteristic parameters, an index indicating a name of the characteristic parameter having a highest level of importance and the highest level of importance may be displayed.

For example, when the SHAP value is specified for each of “positive P-wave amplitude” and “area below positive P-wave” associated with the P-wave portion of the measurement waveform WF and the SHAP value of “positive P-wave amplitude” is higher, only the index corresponding to the SHAP value specified for “positive P-wave amplitude” is displayed in a manner of overlapping the measurement waveform WF.

According to such a configuration, it is possible to prevent a decrease in visibility due to a plurality of indices being displayed in a limited region in the measurement waveform WF.

Alternatively, as illustrated in FIG. 6, the output device 13 may display an index indicating the name of at least one of a plurality of characteristic parameters and the SHAP value specified for the at least one characteristic parameter in a manner of not overlapping the measurement waveform WF of the electrocardiogram.

In this example, the measurement waveform WF of the electrocardiogram corresponding to a plurality of heartbeats is displayed, and an average waveform may be displayed the same or similarly to the example illustrated in FIG. 5. In addition, as illustrated in this example, an index indicating the level of importance specified using gradient-weighted class activation mapping (grad-CAM) may be displayed in a manner of overlapping the measurement waveform WF as necessary. In this example, a band-shaped index is superimposed and displayed on a waveform portion determined to have a high level of importance for the prediction result that “pAF may occur”. The level of importance corresponds to the depth of the color of the band.

According to such a configuration, the validity of the output index can be examined with reference to the measurement waveform WF of the electrocardiogram. In addition, another index indicating the level of importance may be used in combination as necessary.

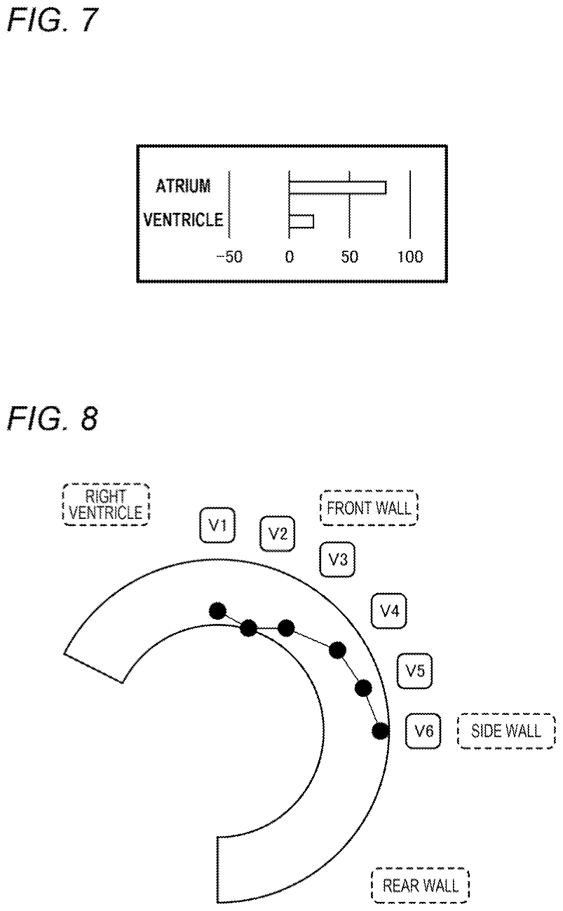

As illustrated in FIG. 7, the output device 13 may display indices in association with an atrium and a ventricle. The atrium and the ventricle are examples of a plurality of body parts of a subject.

Specifically, an index associated with the atrium is generated by adding SHAP values specified for characteristic parameters related to the P-wave of the electrocardiogram. Same or similarly, an index associated with the ventricle is generated by adding SHAP values specified for characteristic parameters related to a QRST-wave of the electrocardiogram.

The addition may be performed for all characteristic parameters, or be performed for a predetermined number of characteristic parameters in descending order of the SHAP values. The multiplication of coefficients may be appropriately performed. In this example, the magnitude of a sum of SHAP values corresponds to the length of a bar extending in the left-right direction.

As illustrated in FIG. 8, the output device 13 may display an index in association with each of leads V1 to V6. FIG. 8 schematically displays a shape of a heart as viewed from below a human body, and indices related to the respective leads V1 to V6 are arranged to correspond to positions in the heart. That is, the positions at which the V1 to V6 leads are acquired are examples of a plurality of body parts of the subject.

Specifically, the index associated with the acquisition position of the V1 lead is generated by adding SHAP values specified for characteristic parameters related to the V1 lead. The same processing is performed for each of the leads V2 to V6. The addition may be performed for all characteristic parameters, or be performed for a predetermined number of characteristic parameters in descending order of the SHAP values. The multiplication of coefficients may be appropriately performed. In this example, the magnitude of a sum of SHAP values corresponds to a distance from the center of the figure imitating the heart. The larger the distance from the center is, the higher the specified level of importance is.

According to the configuration as described above, it is possible to intuitively grasp which characteristic parameter related to a part of the heart contributed to the prediction result of the electrocardiogram. In the example illustrated in FIG. 7, an analysis of a certain suspected abnormality in the atrium may be performed on the prediction result that “pAF may occur”. In the example illustrated in FIG. 8, an analysis of a certain suspected abnormality in a side wall of the heart may be performed on the prediction result that “pAF may occur”.

The processor 122 of the processing device 12 having the above-described functions may be implemented by a general-purpose microprocessor that operates in cooperation with a general-purpose memory. Examples of the general-purpose microprocessor include a CPU, a MPU, and a GPU. Examples of the general-purpose memory include a ROM and a RAM. In this case, the ROM may store a computer program that executes the above-described processing. The ROM is an example of a non-transitory computer-readable medium that stores the computer program. The general-purpose microprocessor specifies at least a part of programs stored in the ROM, loads the program into a RAM, and executes the above-described processing in cooperation with the RAM.

The computer program may be pre-installed in the general-purpose memory, and may be downloaded from an external server via a communication network and be installed in the general-purpose memory. In this case, the external server is an example of the non-transitory computer-readable medium that stores the computer program.

The processor 122 of the processing device 12 having the above-described functions may be implemented by a dedicated integrated circuit that can execute the above-described computer program, such as a microcontroller, an ASIC, or an FPGA. In this case, the above computer program is pre-installed in a storage element provided in the dedicated integrated circuit. The storage element is an example of a computer-readable medium that stores the computer program. The processor 122 of the processing device 12 having the above-described functions may be implemented by a combination of a general-purpose microprocessor and a dedicated integrated circuit.

The above embodiment is merely an example for facilitating understanding of the presently disclosed subject matter. Configurations according to the above embodiment can be appropriately changed and improved without departing from the gist of the presently disclosed subject matter.

When an index to be output by the output device 13 is generated, a local interpremodel-expression expansion (LIME) algorithm may be used instead of the SHAP algorithm.

In the above embodiment, the output device 13 constitutes a part of the electrocardiograph 10. However, when the index data ID can be received from the processing device 12 via wired communication or wireless communication, the output device 13 may be a device provided independently of the electrocardiograph 10.

A part of the processing performed by the reception device 11 in the above embodiment may be executed by the processor 122 of the processing device 12. For example, the processing of acquiring the value PV of each characteristic parameter from the waveform data WD may be performed by the processor 122.

In the embodiment described above, the processing device 12 predicts the presence or absence of the potential of paroxysimal atrial fibrillation for the subject 30. Alternatively, in addition to or instead of the paroxysimal atrial fibrillation, the presence or absence of a potential of developing another cardiac disease may be predicted. Examples of the other cardiac disease include premature atrial contraction, paroxysmal supraventricular tachycardia, premature ventricular contraction, ventricular tachycardia, ventricular fibrillation, and myocardial infarction.

The presence or absence of a potential of developing a disease other than a cardiac disease may be targeted. An example of the other disease includes epilepsy seizure. When the presence or absence of a potential of the epilepsy seizure is predicted, characteristic parameters related to an a wave, a β wave, a θ wave, and the like are acquired based on an electroencephalogram acquired from a subject by an electroencephalograph, and levels of importance of the characteristic parameters for a prediction result are specified. That is, the electroencephalogram is an example of physiological information. The electroencephalograph is an example of a physiological information acquisition apparatus.

Other examples of the disease include hypohypertension and hypertension. When the presence or absence of a potential of hypotension or hypertension is predicted, characteristic parameters such as systolic peak time, diastolic time, and a cardiac output are acquired based on a blood pressure waveform acquired from a subject by a sphygmomanometer, and levels of importance of the characteristic parameters for a prediction result are specified. That is, the blood pressure waveform is an example of physiological information. The sphygmomanometer is an example of a physiological information acquisition apparatus.

Another example of the other disease includes apnea syndrome. When the presence or absence of a potential of developing apnea syndrome is predicted, characteristic parameters such as expiration time, inspiration time, and breathing time are acquired based on a respiratory waveform acquired from a subject by a respirometer, and levels of importance of the characteristic parameters for a prediction result are specified. That is, the respiratory waveform is an example of physiological information. The respirometer is an example of a physiological information acquisition apparatus.

The expression “at least one of two main bodies A and B” used in the present specification for A and B includes a case where A alone is specified, a case where B alone is specified, and a case where both A and B are specified. Each of the main bodies A and B may be singular or plural unless otherwise specified.

The expression “at least one of three main bodies A, B, and C” used in the present specification for A, B, and C includes a case where A alone is specified, a case where B alone is specified, a case where C alone is specified, a case where A and B are specified, a case where B and C are specified, a case where A and C are specified, and a case where all of A, B, and C are specified. Each of the main bodies A, B, and C may be singular or plural unless otherwise specified. The same applies when four or more bodies are described.

What is claimed is:

1. A physiological information acquisition apparatus that acquires physiological information of a subject, the apparatus comprising:
a reception device configured to receive waveform data corresponding to a measurement waveform of the physiological information from a sensor, and to acquire values of a plurality of characteristic parameters associated with the measurement waveform based on the waveform data;
a processing device configured to input the values of the plurality of characteristic parameters to a machine-learned model to acquire a prediction result for at least one of a plurality of classes into which the waveform data is classified, and to specify a level of importance of each of the plurality of characteristic parameters for the prediction result; and
an output device configured to output an index indicating a name of at least one of the plurality of characteristic parameters and the level of importance specified for the at least one characteristic parameter.

2. The physiological information acquisition apparatus according to claim 1,
wherein the index is configured to compare relative levels of importance specified for at least two of the plurality of characteristic parameters.

3. The physiological information acquisition apparatus according to claim 2,
wherein the index indicates the levels of importance specified by using SHapley Additive exPlanations.

4. The physiological information acquisition apparatus according to claim 1,
wherein the output device displays the index in a manner of overlapping the measurement waveform.

5. The physiological information acquisition apparatus according to claim 1,
wherein the output device displays the index in a manner of not overlapping the measurement waveform.

6. The physiological information acquisition apparatus according to claim 5,
wherein the index is displayed in association with each of a plurality of body parts of the subject.

7. The physiological information acquisition apparatus according to claim 4,
wherein the output device displays an index, the index indicating a name of the characteristic parameter having a highest level of importance among the plurality of characteristic parameters associated with a specific portion in the measurement waveform and the highest level of importance, in a position corresponding to the specific portion.

8. The physiological information acquisition apparatus according to claim 1,
wherein the index includes a value of the at least one characteristic parameter.

9. The physiological information acquisition apparatus according to claim 1,
wherein the machine-learned model is generated by machine learning using a neural network.

10. A processing device that processes physiological information of a subject, the device comprising:
an interface configured to receive values of a plurality of characteristic parameters acquired based on waveform data corresponding to a measurement waveform of the physiological information, the plurality of characteristic parameters being associated with the measurement waveform; and
one or more processors configured to input the values of the plurality of characteristic parameters to a machine-learned model to acquire a prediction result of at least one of a plurality of classes into which the waveform data is classified, specify a level of importance of each of the plurality of characteristic parameters for the prediction result, and output index data corresponding to an index indicating a name of at least one of the plurality of characteristic parameters and the level of importance specified for the at least one characteristic parameter.

11. A non-transitory computer readable storage medium that stores a computer program executable by one or more processors mounted on a processing device that processes physiological information of a subject, the computer program causing the processing device to execute processing of:
receiving values of a plurality of characteristic parameters acquired based on waveform data corresponding to a measurement waveform of the physiological information, the plurality of characteristic parameters being associated with the measurement waveform,
inputting the values of the plurality of characteristic parameters to a machine-learned model to acquire a prediction result for at least one of a plurality of classes into which the waveform data is classified,
specifying a level of importance of each of the plurality of characteristic parameters for the prediction result, and
outputting index data corresponding to an index indicating a name of at least one of the plurality of characteristic parameters and the level of importance specified for the at least one characteristic parameter.
